(19) United States
(12) Patent Application Publication
(10) Pub. No.: US 2004/0110281 A1
(43) Pub. Date: Jun. 10, 2004

(54) Vector and a method for expression and selection of random peptide sequences

(76) Inventors: Andreas Meinke, Pressbaum (AT); Tamas Henics, Vienna (AT)

Correspondence Address: Mark B Wilson, Fulbright & Jaworski, Suite 2400, 600 Congress Avenue, Austin, TX, 78701, US

(21) Appl. No.: 10/297,969

(22) PCT Filed: Jun. 12, 2001
(86) PCT No.: PCT/EP01/06617

(30) Foreign Application Priority Data
Jun. 15, 2000 (AT) A 1037/2000

(51) Int. Cl. C12N 15/74

(52) U.S. Cl. 435/320.1

(57) ABSTRACT

The invention relates to an expression/selection vector for ex-pressing random peptide sequences which comprises

three restriction sites RS1, RS2 and RS3, which are unique in the vector, RS3 being located downstream relative to RS1 and upstream relative to RS2,

an insert coding for a random peptide inserted in RS3,

a selection marker gene located downstream of RS2 and

the random peptide sequence and the selection marker gene being expressable in frame to form a fusion protein comprised of the random peptide and the selection marker,

a method for selecting random peptide sequences using such a vector and an expression/selection vector library comprising a library of such vectors.

The invention relates to a vector and a method for expression and selection of random peptide sequences.

Screening of randomized fragments of genomic DNA is one of the key issues of genomics. A specific problem is that only one of three reading frames usually is desired or leads to a suitable expression product when such genomic DNA is screened. Moreover, a DNA to be screened has two possible reading directions where only one is the correct one.

When such random peptide sequences are built into fusion proteins, often leader peptides are provided 5′ to the fusion peptide/protein to be expressed (e.g. if the product has to exert its function in the periplasm or when the protein is supposed to be incorporated into the outer membrane or exported). Furthermore, selection markers are used which are expressed separately or at least as separate proteins in a common operon.

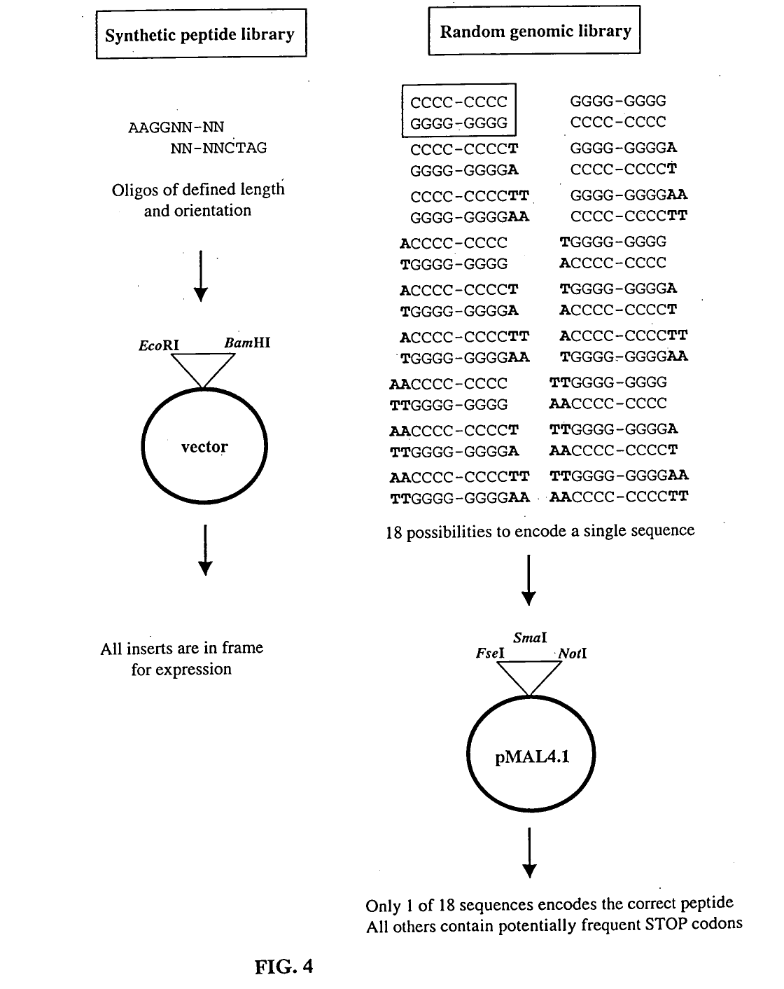

In the case a leader peptide is used for the screening of random peptide sequences together with a displayed carrier protein, eighteen possibilities exist to insert a DNA encoding for a randomized peptide between leader peptide and display peptide (see also FIG. 4). It follows that only one out of eighteen clones shows the right insert. For usual screening methods this is often an unessential problem. However, there are specific screening systems where such circumstances are undesired, especially, if rare members of a library are to be detected or different screening rounds should be performed with different vectors.

Although it is known to combine certain selection markers such as β-lactamase to signal peptides of other proteins to direct the signal markers into the specific locations inside the cell (s. Ghrayheb et al. EMBO J. 3(10) (1984) 2437-2442) such a vector has never been applied in the selection of random peptide sequences.

It is therefore an object of the present invention to provide a vector for expression and selection of random peptide sequences wherein the random peptide sequence is expressed as a fusion protein with the selection marker and wherein only those sequences are selected and expressed which show the correct orientation and the correct reading frame. This object alone is novel and was hitherto unrecognized in the art.

This object is solved by a vector for expression and selection of random peptide sequences which is charcterized in that it comprises three restriction sites RS1, RS2 and RS3, which are unique in the vector, RS3 being located downstream relative to RS1 and upstream relative to RS2,

an insert coding for a random peptide inserted in RS3,

a selection marker gene located downstream of RS2 and

the random peptide sequence and the selection marker gene being expressable in frame to form a fusion protein comprised of the random peptide and the selection marker.

The vector according to the present invention carries the random peptide sequence in a way that it is only selected if the correct reading frame and orientation is given. Moreover, the random peptide sequence may be excised from the vector (by cleaving with RS1 and RS2 restriction enzymes) and transferred to other (cloning) vectors in a defined orientation and reading frame. The present expression/selection vector system is therefore especially suitable for screening of peptides with binding domains which should be expressed (in a later stage) e.g. with the use of bacterial phage display or bacterial display in order to be selected with a given binding partner or a library of binding partners (e.g. antibodies or mixture of antibodies). The present system is especially suited in a screening system as described in the WO 99/30151 e.g. for preparing a suitable library of epitopes. Such binding epitopes may be preselected with the vector according to the present invention and transferred in the right orientation and reading frame to other library vectors. Of course the present vectors may be used for preparing libraries or populations of amplifyable genetic packages used in bacterial or phage display (e.g. as disclosed in EP 0 436 597 B1).

The selection of clones with the correct reading frame reduces the complexity of the library, meaning fewer members have to be screened. For the use of a screening system where it is an absolute necessity to screen only those cells having a desired expression product, the present method is extremely important. For example, in the screening system according to the WO99/30151, only those cells should be screened that do display the epitope on the surface, because cells not expressing the protein on the surface (because e.g. the fusion renders it out-of-frame) cannot be killed by the selection agent.

Preferably, the vector according to the present invention comprises a leader sequence upstream to RS1 suitable for being expressed in frame with the random peptide and the selection marker and suitable for directing the fusion protein comprised of the leader sequence, the random peptide and the selection marker to a predetermined location. The leader sequence may direct the expressed fusion protein into desired cell compartment (in an eukaryotic cell) or to specific membrane location (e.g. to the outer membrane or the periplasm in prokaryotic cells). The leader sequence may be the natural leader sequence for the selection marker, or according to a preferred embodiment, an established and powerful leader sequence heterogeneous for the selection marker used.

Preferably, RS1 and RS2 are restriction sites which are rare and may e.g. be selected specifically for the organism from which the random peptide sequences are derived from (e.g. a restriction site which is rare or absent in the genome of this organism). According to a preferred embodiment rare cutting enzymes sites are provided as RS1 and RS2. Especially 8 bp cutter sites are preferred as RS1 and RS2, such as AscI, FseI, NotI, PmeI, SbfI, SfiI, PacI or SgrAI sites. RS3 is preferably a site which directly or indirectly leads to blunt ends after cutting (e.g. SmaI).

It is preferred, especially in case of blunt cut of RS3, that the vector without random peptide sequence insert is not in frame with respect to the selection marker so that upon religation without incorporation of a random peptide insertion the selection marker is not expressed by the vector. The cloning could also be done using for example a restriction site leaving an overhang in the vector in combination with the ligation of respective linkers to the inserts.

The random peptide sequence is preferably derived from the genome of an organism, especially from the genome of a pathogen. The derivation process is preferably a random cutting with a frequently cutting enzyme or the generation of random DNA fragments by DNAseI, optionally with adaptations to the restriction overlaps (e.g. blunt making, introducing sticking ends, linker addition, etc.). Another preferred derivation process comprises the mechanical breaking of the genomic DNA, including sonication or nebulisation, into DNA molecules with appropriate size. Preferably the sequence inserted in the vector has a length of 20 to 500 bp, preferably 100 to 300 bp. Inserts which are longer or shorter may also be provided, however, the risk that the expression efficiency decreases is given with such inserts. Of course, also cDNA libraries, ESTs, etc. may be introduced into the vector as random peptide sequences.

The genome wherefrom the random peptides sequences are derived from are preferably from viral pathogens, especially from HAV, HBV, HCV, HIV-1, HIV-2, EBV, HTLV-I or HTLV-II from a bacterial pathogen, especially from S. aureus, M. tuberculosis, C. pneumoniae, S. typhimurium, Y. pestis, S. epidermidis or from a eukaryotic pathogen, especially from T. brucei.

It is also possible to provide in frame linker sequences between the random peptide sequence and the selection marker gene as well as between the leader peptide sequence and the random peptide sequence.

The selection marker may be any selection marker used and suitable in the art, preferably an antibiotic resistance is used, such as kanR, CanR, Zeocin, Neomycin etc.

According to a preferred embodiment of the present invention β-lactamase is used as a selection marker optionally in combination with an OmpA or Lpp leader sequence.

According to another aspect the present invention is drawn to a method for selecting random peptide sequences comprising the following steps:

providing a vector comprising three restriction sites RS1, RS2 and RS3, which are unique in the vector, RS3 being located downstream relative to RS1 and upstream relative to RS2, a selection marker gene located downstream of RS2 and optionally a leader sequence being located upstream of RS1,

inserting a library of random peptide sequences into RS3 to create a vector library,

introducing the vector library into a suitable host, which is capable of expressing a fusion protein comprised of random peptide and selection marker, to create a host library,

cultivating said host library on a medium selective with respect to the selection marker, thereby selecting the host individuals which express said fusion protein.

This method results in a library of selected vectors which had a clearly defined reading frame and orientation whereby the random peptide sequence may be excised in a way that the defined orientation and reading frame is preserved.

According to a preferred embodiment of the method according to the present invention the vector of the selected host individual or a (sub-)library of vectors of a selected host library is isolated and cut with RS1 and RS2 cutting restriction enzymes to obtain a fragment containing the random peptide sequence in a defined reading frame and orientation.

This RS1/RS2 fragment may be inserted into another vector which has been cut with RS1 and RS2 cutting restriction enzymes (to obtain an RS1 and RS2 insertion site) and—after insertion of the RS1/RS2 random peptide sequence fragment—is suitable for expressing this random peptide in a defined way.

According to a preferred embodiment of the method according to the present invention the cultivation medium wherein the host library is cultivated contains an antibiotic and the selection marker is an antibiotic resistance. Preferred antibiotic/antibiotic resistance pairs are β-lactamase—ampicillin, aminoglycoside phosphotransferase (acetyltransferase, nucleotidyltransferase) —kanamycin (neomycin), chloramphenicol acetyltransferase—chloramphenicol, Tet^{R}-Tn10 gene product-tetracycline and Sh ble gene product—zeocin.

According to another aspect of the present invention the invention also relates to a library of vectors (i.e. a variety of vectors with different random peptide sequences) according to the present invention comprising a library of random peptide sequences inserted in RS3, i.e. the invention is also drawn to a library of vectors according to the present invention or cells containing such vectors.

According to another aspect, the present invention relates to a system of vectors comprising a vector according to the present invention and a (second) vector wherein the RS1/RS2 fragment (insert) of this vector may be inserted and preferably expressed. This second vector may be an efficient expression vector, especially designed for producing large quantities of the RS1/RS2 fragment encoded polypeptide. Transfer of the RS1/RS2 fragment from the vector according to the present invention into the second vector is straight forward, because reading frame and orientation of the fragment are clearly identified by the selection method according to the present invention.

The invention will be explained in more detail by way of the following example and the associated drawing figures to which, however, it shall not be restricted.

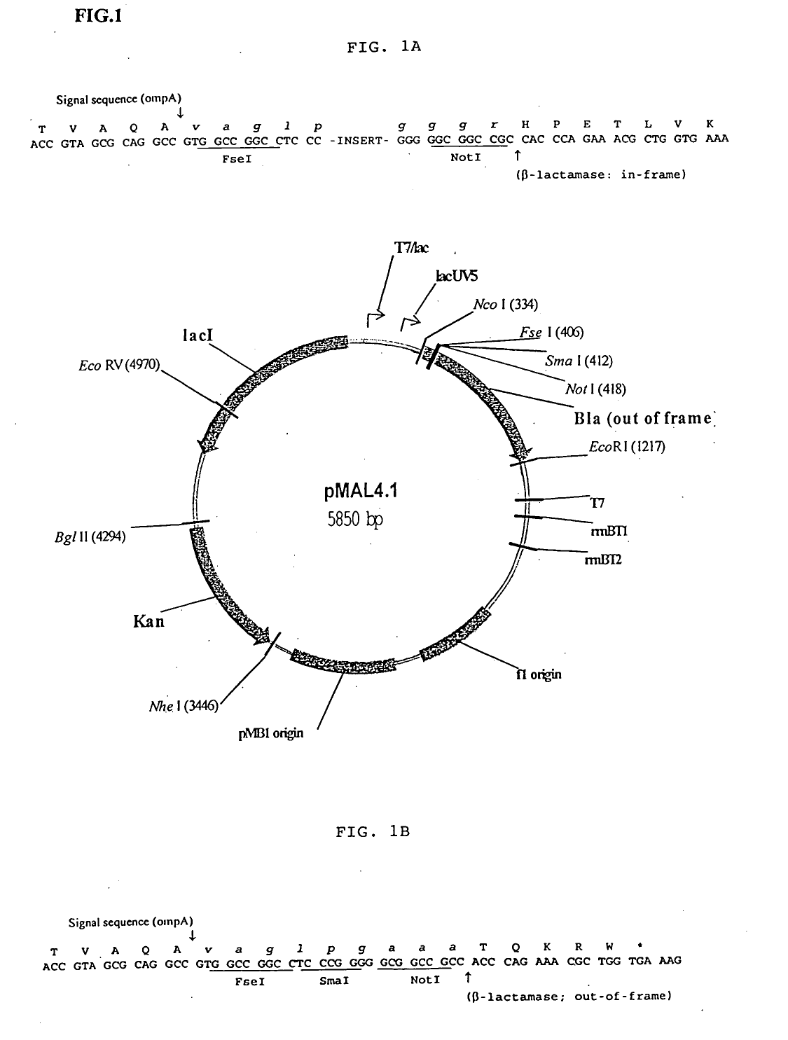

FIG. 1 shows plasmid pMAL4.1 (1A) and the insertion site in the β-lactamase gen (1B);

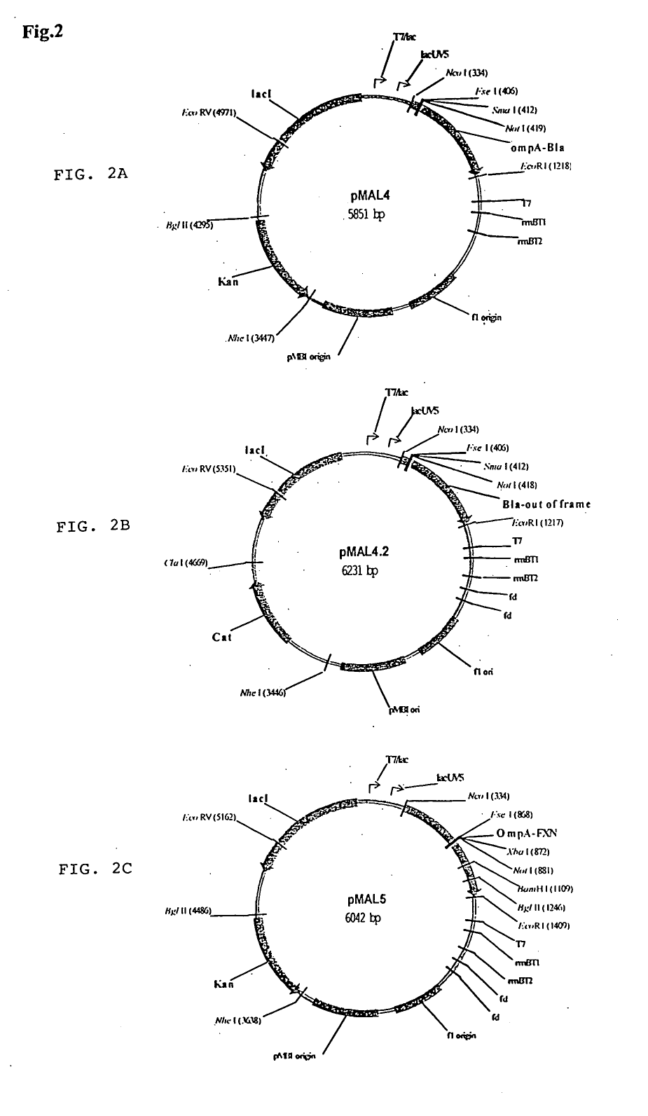

FIGS. 2A-C shows the plasmids for library construction;

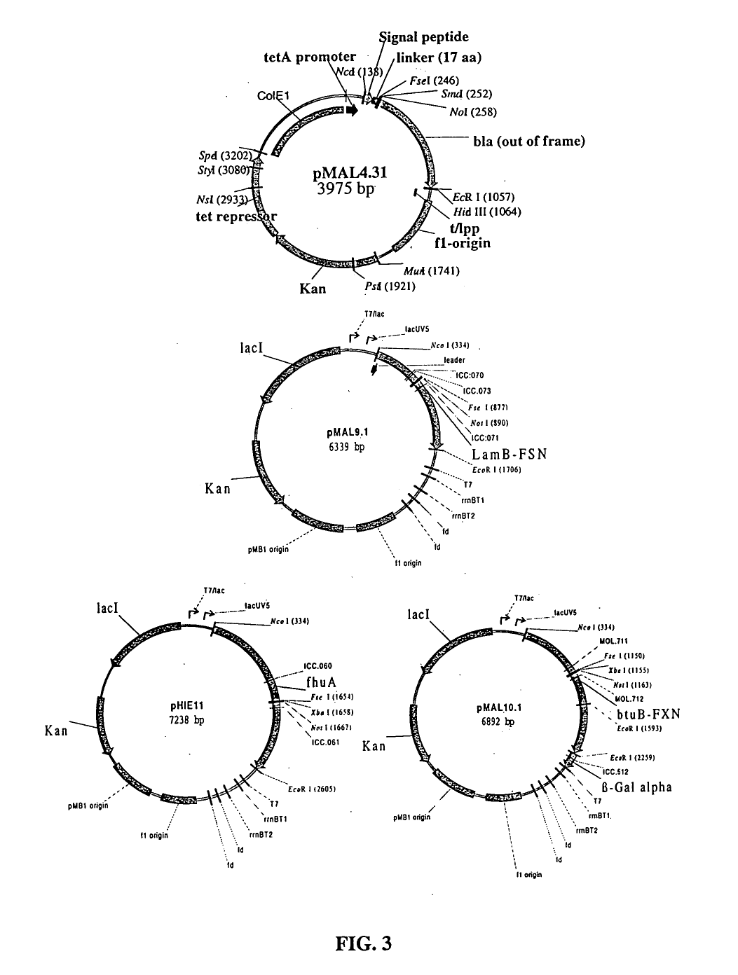

FIG. 3 shows the generation of a library of S. epidermidis;

FIG. 4 shows a graphic representation of the advantages of the present invention in comparison with library screening techniques according to the prior art.

### EXAMPLES

### Example 1

### Generation of a Library of S. aureus

A plasmid is generated according to FIG. 1 which allows blunt end insertion of random generated DNA fragments into a linker situated between the OmpA leader peptide and the mature β-lactamase gene. The plasmid if religated at the blunt end restriction site leads to an out-of-frame β-lactamase gene. A series of vectors has been designed (pMAL4, pMAL4.1, pMAL4.2, pMAL5) which contain FseI/NotI (as RS1 and RS2 sites) and a SmaI site (pMAL4, pMAL4.1) or a XbaI (pMAL5) as an RS3 site (see FIG. 2).

A library from S. aureus is inserted into the SmaI site of pMAL4.1.

Insertions that lead to an out of frame β-lactamase gene can be eliminated by their sensitivity against ampicillin. The inserted DNA fragments can be excised with two flanking restriction sites (in the present case FseI and NotI) that will allow the insertion of these fragments in the same orientation as in the original plasmid.

Protocol for pMAL4.1 Library Construction Vector Preparation

25 μg plasmid DNA

5 μl 10× buffer (NEB 4)

5 μl SmaI (20 U/μl; NEB)

add H₂O (Merck, HPLC grade) to 50 μl

Incubate for 2-5 h at 25° C., check digest on agarose gel Heat-inactivate SmaI for 20 min at 65° C.

Separate on 1% agarose gel (GTG-agarose, FMC) in 1×TAE buffer (TBE buffer)

Elute the vector from the gel using a gel extraction kit (Qiagen) Transform aliquot into DH10B cells via electroporation to test digest

dt-Tailing of Vector

25 μl digested plasmid

8 μl 25 mM MgCl₂ (Perkin Elmer)

5 μl 10×Stoffel buffer (Perkin Elmer)

2 μl 100 mM dTTP (Gibco)

1 μl Stoffel fragment of Taq polymerase (10 U/μl; Perkin Elmer) add H₂O (Merck, HPLC grade) to 50 μl

Incubate at 74° C. for 30 min

Separate on 1% agarose gel (GTG-agarose, FMC) in 1×TAE buffer (TBE buffer)

Elute the vector from the gel using a gel extraction kit (Qiagen) Check efficiency by religation of vector and transformation into DH 10B cells via electroporation

Insert Preparation

Generation of Small (50-60 bp) Fragments

˜25 μg genomic DNA in 50 μl H₂O

5 μl of 10×DNase I buffer (0.5 M Tris-HCl pH 7.5, 0.5 mg/ml BSA)

5 μl of 10×MnCl₂(100 mM)

1 μl of ds qualified DNase 1(2 U/μl)

Incubation at RT for 3 min

Check on 2% GTG-TAE agarose gel

Bulk separate in 2% GTG-TAE agarose gel

Excise appropriate size range (enrichment of fragments are around 50-60 bp range)

Generation of Large Fragments (200-400 bp)

˜125 ˜l genomic DNA of S. aureus in 60 ˜l 1×TM buffer (50 mM Tris-HCl pH 8, 15 mM MgCl₂)

Sonicate in Eppendorf tubes in ice water for 30-40 pulses (100% output, 0.10 s/pulse)

Check fragmentation and size range distribution on 2% GTG-TAE agarose gel

Bulk separate on 2% GTG-TAE agarose gel

Excise fragments and proceed with end treatment for cloning

dA-Tailing of Inserts

Use da-tailing kit from Novagen:

1 μg DNA

8.5 μl 10×dA Tailing buffer (Novagen)

0.5 μl Tth DNA polymerase (2.5 U/μl, Novagen)

add H₂O (Merck, HPLC grade) to 85 μl

Incubate the reaction at 70° C. for 15 min

Extract with 1 volume CIAA (Sigma)

Vortex vigorously for 60 sec, centrifuge at 12,000 g for 1 min Store at −20° C.

Ligation

30 μl ligation reaction:

50 ng vector DNA

100 ng insert DNA

6 μl 5×ligation buffer (Gibco)

3 μl of T4 DNA ligase (1 U/μl, Gibco)

add H₂O (Merck, HPLC grade) to 30 μl

Incubate ligation reaction over night at 16° C.

Precipitate ligation reaction with EtOH as following:

30 μg ligation reaction

70 μl H₂O (Merck, HPLC grade)

10 μl 3M Na-Acetate pH 5.2

1 μl 20 mg/μl glycogen (Roche, molecular biology grade)

200 μl EtOH (Merck, molecular biology grade)

incubate for at least 60 min at −20° C.

suspend pellet in 10 μl H₂O (Merck, HPLC grade)

From the 30 μl ligation, 1/10th of the reaction is used in one transformation.

Transformation

Cells: DH10B (Gibco), DH5a: (Gibco)

Volume of cells: 20 μl/transformation

Cuvette: 0.1 cm gap width, BioRad

BioRad E. coli Genepulser

Voltage: 2.0 kV

Usual time constant: 5.0

Recovery medium: S.O.C.; add to cuvette immediately after pulse Recovery time: 45 min

Plate and grow on LB plates containing 50 μg/ml Kanamycin and Ampicillin

### Protocol for Transfer of FseI/NotI Fragments from pMAL4.1 Library to pMAL5

Vector Preparation

25 μg plasmid DNA

5 μl 10×buffer (NEB 2)

5 μl Fsel (2 U/μl; NEB), 2 μl NotI (10 U/μl; NEB)

add H₂O (Merck, HPLC grade) to 50 μl

Incubate over night at 37° C., check digest on agarose gel

Heat-inactivate enzymes for 20 min at 65° C.

Separate on 1% agarose gel (GTG-agarose, FMC) in 1×TAE buffer (TBE buffer)

Elute the vector from the gel using a gel extraction kit (Qiagen) Transform aliquot into DH10B cells via electroporation to test digest

Insert Preparation

25 μg pMAL4.1 library DNA

5 μl 10× buffer (NEB2)

5 μl FseI (2 U/μl; NEB), 2 μl NotI (10 U/μl; NEB)

add H₂O (Merck, HPLC grade) to 50 μl

Incubate for 2-15 h at 25° C., check digest on agarose gel

Heat-inactivate enzymes for 20 min at 65° C.

Separate on 1% agarose gel (GTG-agarose, FMC) in 1×TAE buffer (TBE buffer)

Elute the vector from the gel using electroelution (Biotrap: Schleicher & Schuell)

Precipitate with ethanol (as for ligation reaction).

Ligation and transformation as for pMAL4.1 library construction.

Plate and grow on LB plates containing 50 μg/ml Kanamycin.

Genomic S. aureus library:

Number of cells on Kan (complexity of library before selection): 1.6×10⁷

Number of cells on Kan/Amp (complexity of library after selection): 1×10⁶

Number of clones tested for insert: 148.

Number of clones with insert in-frame: 148

Number of clones without insert: 0

Number of clones with insert out-of-frame: 0

This shows that only those clones will be selected that do possess an insert in the correct frame.

### Example 2

### Generation of a Library of S. epidermidis Using Vector pMAL4.31.

A plasmid is generated according to FIG. 3 that allows blunt end insertion of random generated DNA fragments into the SmaI site situated between the OmpA leader peptide followed by a linker of 17 amino acids (HPETLVKVKDAEVAGLP) and the mature β-lactamase gene. Any peptide encoded by the library will therefore be expressed with an extra 17 amino acids at the N terminus in pMAL4.31 as compared to 5 amino acids in pMAL4.1. The additional sequence will increase the likelihood that most fusion proteins encoded by the library are delivered to the periplasm, since it was reported that the net charge of the first 18 amino acids of the mature sequence may affect correct translocation across the cytoplasmic membrane (A. V. Kajava et al., 2000, J. Bacteriol. 182:2163-9). All other features of pMAL4.31 are the same as described for pMAL4.1.

In addition, three plasmids have been constructed to accept inserts that were frame selected by either pMAL4.1 or pMAL4.31 while maintaining the correct reading frame when cloned via the FseI and NotI restriction sites. pMAL9.1 encodes the lamB gene, pMAL10.1 the btuB gene and pHIE11 the fhuA gene for display of peptide inserts on the bacterial surface.

S. epidermidis genomic DNA has been fragmented to a size of approximately 70 bp. The genomic fragments have subsequently been ligated to SmaI digested pMAL4.31 and clones have been selected on LB plates containing 50 μg/ml kanamycin only or 50 μg/ml kanamycin and 50 μg/ml ampicillin.

Number of cells on Kan (complexity of library before selection): 3×10⁷

Number of cells on Amp and Kan (complexity of library after selection): 2×10⁶

Number of clones tested for insert: 362

Number of clones with insert in frame: 356

Number of clones without insert: 0

Number of clones with insert out-of-frame: 6

The 6 clones that were determined to have a sequence out-of-frame were subsequently not tested for resistance on ampicillin, since they were not preserved during the time required for the sequence analysis. It is therefore entirely possible that the determined sequence is erroneous. In general, it can be stated that more than 98% of all tested clones show the correct reading frame.

1. Vector for expressing and selecting random peptide sequences of a genome of an organism, characterized in that it comprises
three restriction sites RS1, RS2 and RS3, which are unique in the vector, RS3 being located downstream relative to RS1 and upstream relative to RS2,
an insert coding for a random peptide inserted in RS3, said insert being a fragment with a length of 20 to 500 bp of said genome,
a selection marker gene located downstream of RS2 and
the random peptide sequence and the selection marker gene being expressable in frame to form a fusion protein comprised of the random peptide and the selection marker.

2. Vector according to claim 1, characterized in that it comprises a leader sequence upstream to RS1 suitable for being expressed in frame with the random peptide and the selection marker and suitable for directing the fusion protein comprised of the leader sequence, the random peptide and the selection marker to a predetermined location.

3. Vector according to claim 1 or 2, characterized in that RS1 and RS2 are 8 bp cutting restriction enzyme sites.

4. Vector according to any one of claims 1 to 3, characterized in that cutting RS3 results in blunt ends.

5. Vector according to any one of claims 1 to 4, characterized in that the random peptide sequence is derived from the genome of a pathogen.

6. Vector according to any one of claims 1 to 5, characterized in that the random peptide sequence has a length of 100 to 300 bp.

7. Vector according to any one of claims 1 to 6, characterized in that it comprises an in frame linker sequence between the random peptide sequence and the selection marker gene.

8. Vector according to any one of claims 1 to 7, characterized in that the selection marker is an antibiotic resistance.

9. Vector according to any one of claims 1 to 8, characterized in that the selection marker is β-lactamase.

10. Vector according to any one of claims 1 to 9, characterized in that it contains an OmpA leader sequence.

11. Vector according to any one of claims 1 to 10, characterized in that the random peptide sequence is derived from a viral pathogen.

12. Vector according to any one of claims 1 to 11, characterized in that the random peptide sequence is a fragment of HAV, HBV, HCV, HIV-1, HIV-2, EBV, HTLV-I or HTLV-II.

13. Vector according to any one of claims 1 to 10, characterized in that the random peptide sequence is derived from a bacterial pathogen, especially from S. aureus, M. tuberculosis, C. pneumoniae, S. typhimurium, Y. pestis or S. epidermidis.

14. Vector according to any one of claims 1 to 10, characterized in that the random peptide sequence is derived from a eukaryotic pathogen, especially from T. brucei.

15. Method for selecting random peptide sequences of a genome of an organism comprising the following steps:
providing a vector comprising three restriction sites RS1, RS2 and RS3, which are unique in the vector, RS3 being located downstream relative to RS1 and upstream relative to RS2, and a selection marker gene located downstream of RS2 and optionally a leader sequence being located upstream of RS1,
inserting a library of random peptide sequences into RS3 to create a vector library, said inserts being fragments with a length of 20 to 500 bp of said genome,
introducing the vector library into a suitable host, which is capable of expressing a fusion protein comprised of random peptide and selection marker, to create a host library,
cultivating said host library on a medium selective with respect to the selection marker, thereby selecting the host individuals which express said fusion protein.

16. Method according to claim 15, characterized in that the vector of the selected host individuals is isolated and cut with RS1 and RS2 cutting restriction enzymes to obtain a fragment containing the random peptide sequence in a defined reading frame and orientation.

17. Method according to claim 16, characterized in that the fragment is inserted into another vector which has been cut with RS1 and RS2 cutting restriction sites to obtain an RS1/RS2 insertion site.

18. Method according to any one of claims 15 to 17, characterized in that the cultivation medium contains an antibiotic and the selection marker is an antibiotic resistance.

19. Expression/selection vector library characterized in that in a vector according to claims 1 to 14 a library of random peptide sequences is inserted.

20. Vector system comprising a vector according to any one of claims 1 to 14 and a second vector comprising an RS1/RS2 insertion site.

21. Vector system according to claim 20, characterized in that said second vector is an expression vector allowing expression of DNA fragments inserted in said RS1/RS2 insertion site.


SEQUENCE LISTING (19 sequences)

SEQ ID NO: 1 - protein, 17 aa, Artificial Sequence
  Description of Artificial Sequence  Synthetic Peptide
HPETLVKVKDAEVAGLP

SEQ ID NO: 2 - protein, 10 aa, Artificial Sequence
  Description of Artificial Sequence  Synthetic Peptide
TVAQAVAGLP

SEQ ID NO: 3 - DNA, 29 bp, Artificial Sequence
  Description of Artificial Sequence  Synthetic Primer
accgtagcgcaggccgtggccggcgtccc

SEQ ID NO: 4 - protein, 11 aa, Artificial Sequence
  Description of Artificial Sequence  Synthetic Peptide
GGGRHPETLVK

SEQ ID NO: 5 - DNA, 33 bp, Artificial Sequence
  Description of Artificial Sequence  Synthetic Primer
gggggcggccgccacccagaaacgctggtgaaa

SEQ ID NO: 6 - protein, 19 aa, Artificial Sequence
  Description of Artificial Sequence  Synthetic Peptide
TVAQAVAGLPGAAATQKRW

SEQ ID NO: 7 - DNA, 63 bp, Artificial Sequence
  Description of Artificial Sequence  Synthetic Primer
accgtagcgcaggccgtggccggcctcccgggggcggccgccacccagaaacgctggtgaaag

SEQ ID NO: 8 - DNA, 10 bp, Artificial Sequence
  Description of Artificial Sequence  Synthetic Primer
cccccccctt

SEQ ID NO: 9 - DNA, 10 bp, Artificial Sequence
  Description of Artificial Sequence  Synthetic Primer
ggggggggaa

SEQ ID NO: 10 - DNA, 10 bp, Artificial Sequence
  Description of Artificial Sequence  Synthetic Primer
acccccccct

SEQ ID NO: 11 - DNA, 10 bp, Artificial Sequence
  Description of Artificial Sequence  Synthetic Primer
tgggggggga

SEQ ID NO: 12 - DNA, 11 bp, Artificial Sequence
  Description of Artificial Sequence  Synthetic Primer
acccccccctt

SEQ ID NO: 13 - DNA, 11 bp, Artificial Sequence
  Description of Artificial Sequence  Synthetic Primer
tggggggggaa

SEQ ID NO: 14 - DNA, 10 bp, Artificial Sequence
  Description of Artificial Sequence  Synthetic Primer
aacccccccc

SEQ ID NO: 15 - DNA, 10 bp, Artificial Sequence
  Description of Artificial Sequence  Synthetic Primer
ttgggggggg

SEQ ID NO: 16 - DNA, 11 bp, Artificial Sequence
  Description of Artificial Sequence  Synthetic Primer
aacccccccct

SEQ ID NO: 17 - DNA, 11 bp, Artificial Sequence
  Description of Artificial Sequence  Synthetic Primer
ttgggggggga

SEQ ID NO: 18 - DNA, 12 bp, Artificial Sequence
  Description of Artificial Sequence  Synthetic Primer
aacccccccctt

SEQ ID NO: 19 - DNA, 12 bp, Artificial Sequence
  Description of Artificial Sequence  Synthetic Primer
ttggggggggaa